(19) United States
(12) Patent Application Publication
(10) Pub. No.: US 2005/0089451 A1
(43) Pub. Date: Apr. 28, 2005

(54) Structure of a light-catalyst equipped electric fan

(76) Inventor: Ping Huang, Tainan Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/690,602

(22) Filed: Oct. 23, 2003

(51) Int. Cl. A61L 9/16

(52) U.S. Cl. 422/121; 422/122

(57) ABSTRACT

A light-catalyst equipped electric fan includes an adjustable holding part, a light tube secured to the holding part for producing ultraviolet light, a first motor disposed behind the holding part, a fan blade connected with the first motor, a second motor, a filtering honeycomb having titanium dioxide applied over it, and two joined protecting nets arranged at a front of the electric fan, and connected with an output shaft of the second motor; the filtering honeycomb is disposed between the protecting nets, and stuck on a rear side of a front one of the nets; thus, when the motors and the light tube are activated, air will pass through the filtering honeycomb, and ultraviolet light will shine on the titanium dioxide, and in turns, air passing through the filtering honeycomb is purified, and germs in the air killed.

### BACKGROUND OF THE INVENTION

1. Field of the Invention

The present invention relates to a light-catalyst equipped electric fan, more particularly one, which includes a light tube, and a filtering honeycomb having titanium dioxide (TiO₂) applied over it so that when the electric fan is powered, the TiO₂ will meet ultraviolet light from the light tube to effect chemical reaction of air therewith, which reaction can get rid of bad odor, and decompose detrimental substances in the air.

2. Brief Description of the Prior Art will

When titanium dioxide (TiO₂), a light catalyst, meets ultraviolet light, it will be excited by the photons, and effect chemical reaction of air therewith so that detrimental substances in the air are decomposed into non-harmful ones. With such reaction, germs in the air can be killed, unpleasant odor got rid of, and exhaust of cars and factories decomposed to provide fresh and clean air. TiO₂ has been applied for such purpose on some kinds of home appliances, e.g. air filters, electric lamps, air conditioners, and electric fans, to improve the quality of air in the environment for the health of people.

Referring to FIG. 7, a conventional light-catalyst equipped electric fan is comprised of a rear protecting net 10, and a light-catalyst equipped light tube 20 secured to an inward side of the rear protecting net 10 with fasteners 101. The light-catalyst equipped light tube 20 includes a glass fiber tube, and a light tube disposed in the glass fiber tube. The glass fiber tube has a film of light catalyst, e.g. titanium dioxide, formed thereon while the light tube can emit ultraviolet light. Thus, air passing through the fan is purified, when the electric fan is activated for ultraviolet light to be emitted from the light tube and shine on the light catalyst film over the glass fiber tube.

However, the above light-catalyst equipped electric fan is found to have disadvantages as followings:
1. It takes high cost to manufacture the materials for the light-catalyst equipped light tube 20.
2. The light catalyst film will lose effect after certain length of time of use, and the whole light tube 20 needs to be replaced with a new one. Therefore, using such light tube 20 is a waste of money.
3. The whole outer shell of the electric fan has to be first removed so that the light tube 20 can be replaced with a new one. Therefore, this light-catalyst equipped electric fan is not convenient to use.

### SUMMARY OF THE INVENTION

It is a main object of the present invention to provide a light-catalyst equipped electric fan to overcome the above disadvantages.

The electric fan of the present invention includes a holding part, a light tube secured to the holding part for producing ultraviolet light, a first motor disposed behind the holding part, a fan blade connected with the first motor, a second motor, a filtering honeycomb having titanium dioxide, a light-catalyst, applied over it, and two joined protecting nets arranged at a front of the electric fan. The filtering honeycomb is disposed between the protecting nets, and stuck on a rear side of a front one of the nets while the front protecting net is connected with an output shaft of the second motor. Thus, when the motors and the light tube are activated, air will pass through the filtering honeycomb, and ultraviolet light will shine on the titanium dioxide. And, the filtering honeycomb alone can be replaced with a new one when the light-catalyst applied over it loses effect after certain length of time of use.

### BRIEF DESCRIPTION OF THE DRAWINGS

This invention will be better understood by referring to the accompanying drawings, wherein:

FIG. 1 is an exploded perspective view of the light-catalyst equipped electric fan according to the present invention,

FIG. 2 is a partial exploded perspective view of the light-catalyst equipped electric fan according to the present invention,

FIG. 3 is a side view of the light-catalyst equipped electric fan according to the present invention,

FIG. 4 is a partial vertical section of the light-catalyst equipped electric fan according to the present invention,

FIG. 5 is a perspective view of the light-catalyst equipped filtering honeycomb of the electric fan of the present invention,

FIG. 6 is a front view of the light-catalyst equipped electric fan according to the present invention, and

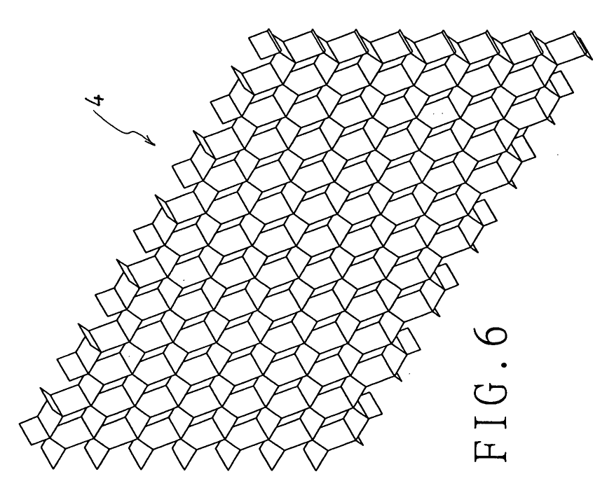

FIG. 7 is an exploded perspective view of the conventional light-catalyst equipped electric fan as described in the Background.

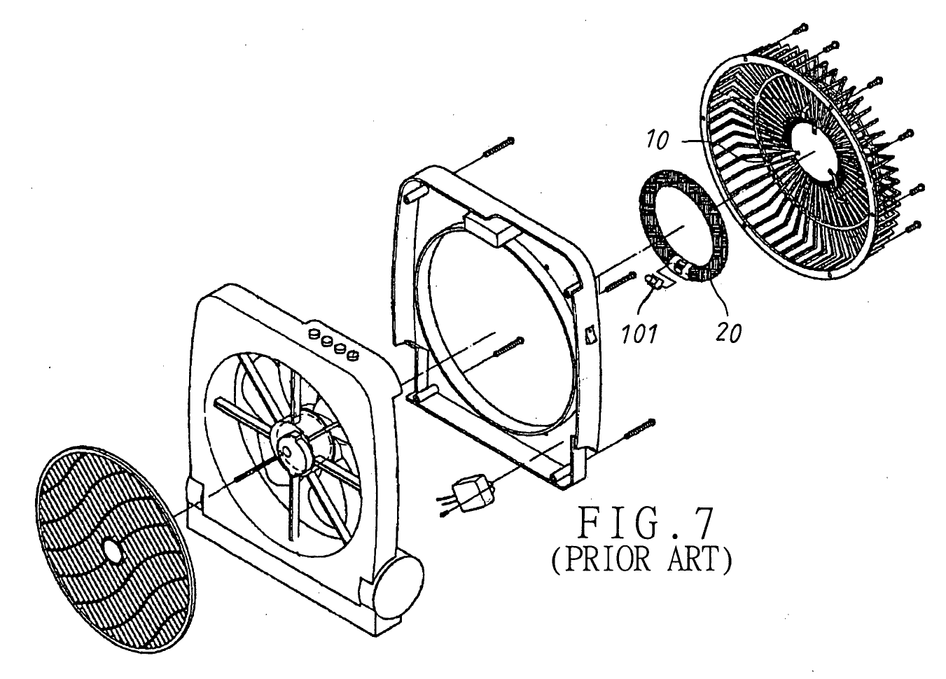

### DETAILED DESCRIPTION OF THE PREFERRED EMBODIMENTS

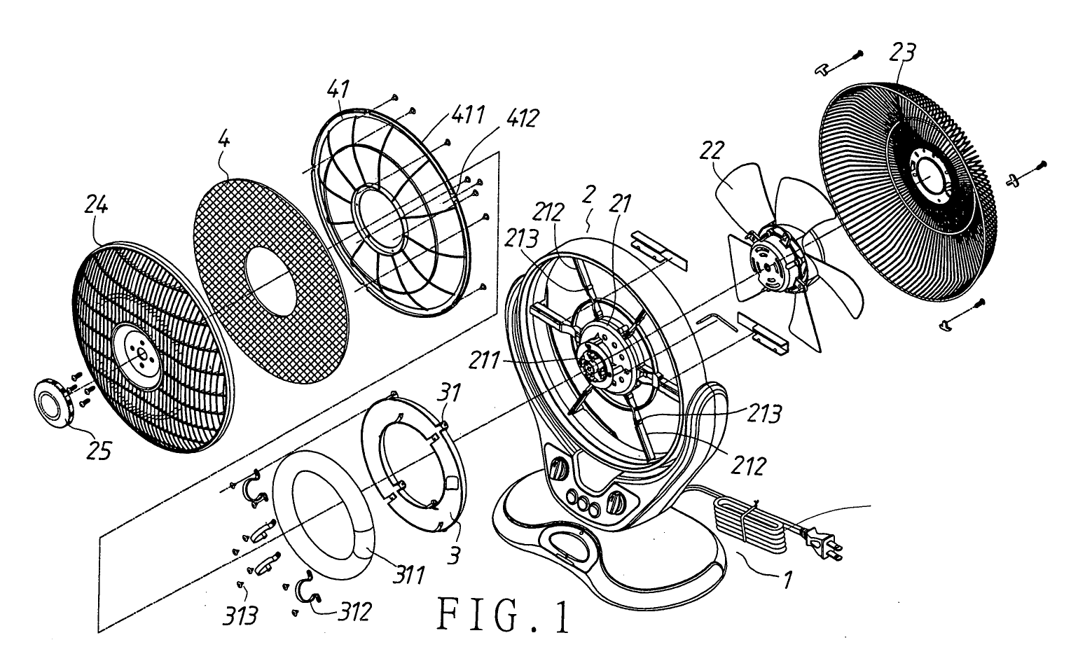

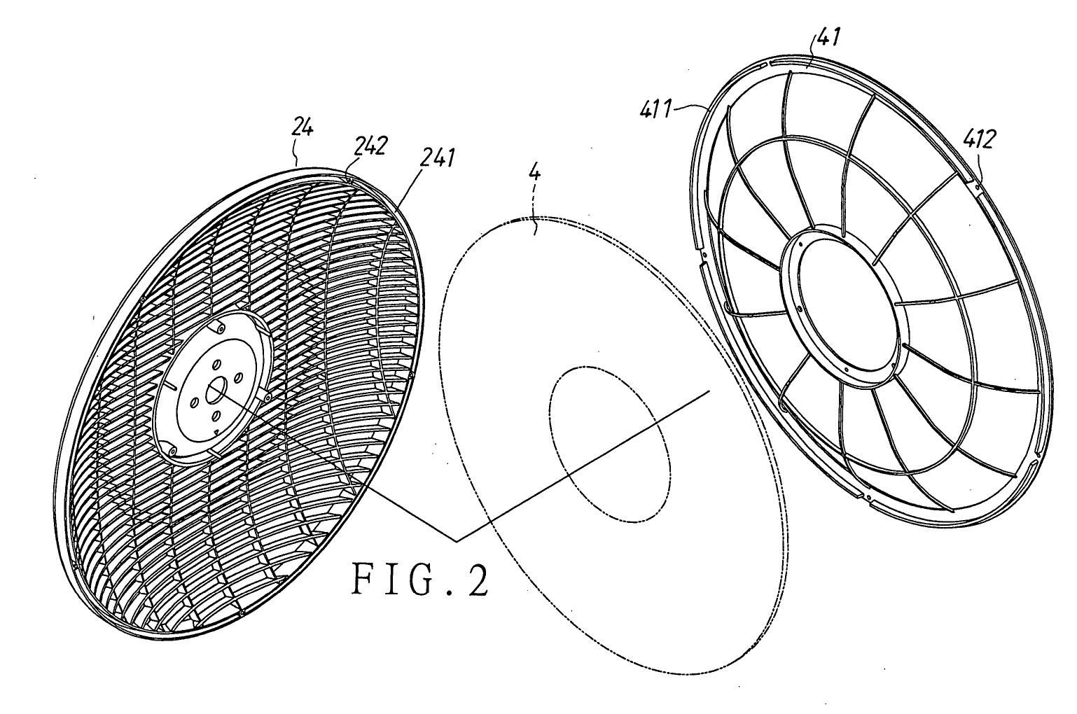

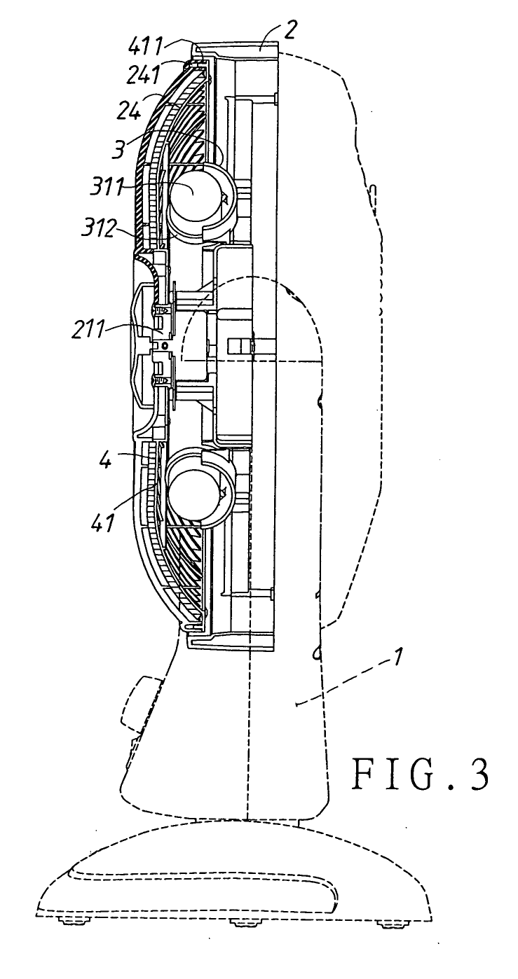

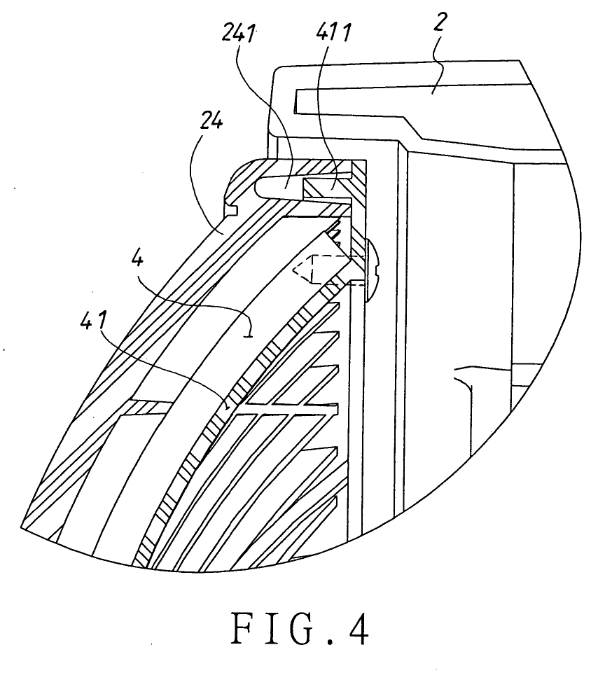

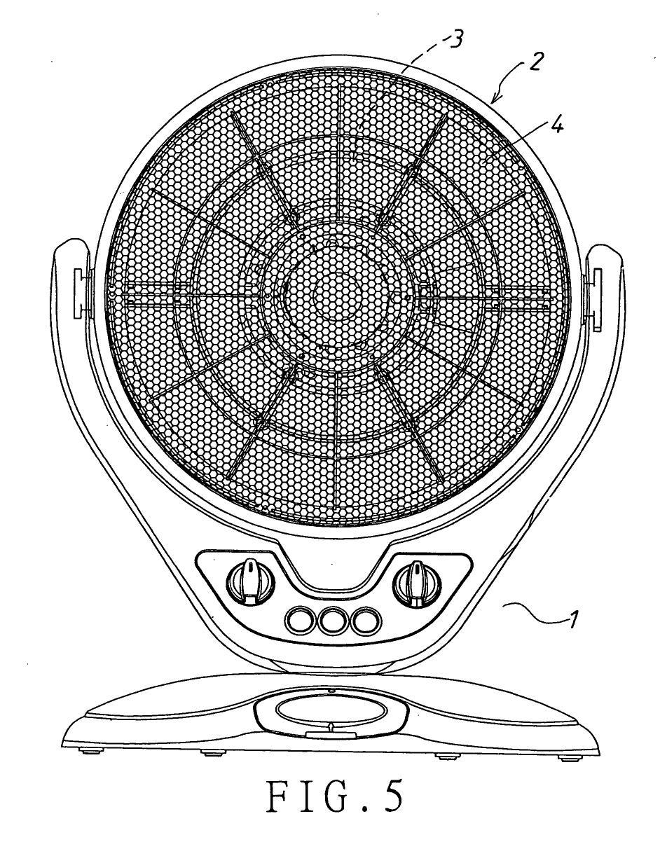

Referring to FIGS. 1 to 5, a preferred embodiment of a light-catalyst equipped electric fan in the present invention includes a main body 1, a holding part 2, a seat 3, a light tube 311, and a filtering honeycomb 4.

The holding part 2 is supported on the main body 1, and can be adjusted in position. The holding part 2 has a locating portion 21, which has ribs 212 arranged in a radial pattern. The ribs 212 have screw holes 213 formed thereon. A first motor (not numbered) is secured to a rear side of the locating portion 21, and a fan blade 22 is connected to an output shaft of the first motor. And, a second motor is secured to the locating portion 21 while a rotary output shaft 211 is connected to an output shaft of the second motor to be turned together therewith. Furthermore, a rear protecting net 23 is disposed on the rear side of the holding part 2.

The seat 3 is provided for holding the light tube 311 in position, and is secured to the locating portion 21 of the holding part 2; the seat 3 has an annular concavely curved portion on one side, and several through holes 3, and the light tube 311 is disposed on the annular concavely curved portion, and secured to the seat 3 by means of fasteners 312, which are disposed over the light tube 311, and which are secured to the seat 3 by screws 313 screwed through the fasteners 312, the through holes 31 of the seat 3, and the screw holes 213 of the ribs 212. In other words, the light tube 311 is secured to the locating portion 21 together with the seat 3.

The filtering honeycomb 4 is made of paper, and has titanium dioxide (TiO₂) applied over it so as to be used as light catalyst. Protecting nets 24, and 41 are respectively disposed on front and rear sides of the filtering honeycomb 4 to protect the filtering honeycomb 4, and the filtering honeycomb 4 is stuck to the rear side of the front protecting net 24. The front protecting net 24 has an annular trench 241 on the edge of a rear side, and screw holes 242 spaced apart along the annular trench 241 while the inner protecting net 41 has protrusions 411 on the edge of a front side thereof, and through holes 412 between the protrusions 411. The protrusions 411 of the inner protecting net 41 is inserted in the trench 241 of the front protecting net 24, and screws (not numbered) are screwed through the holes 241, 412, and screwed into nuts (not numbered); thus, the filtering honeycomb 4 is protected from getting damaged. In addition, an ornamental cap 25 is secured to a center of the front protecting net 24. The protecting nets 24 and 41 are arranged over the front side of the holding part 2, and the rotary output shaft 211 is connected to the ornamental cap 25 as well as the middle of the protecting net 24 so that the protecting nets 24, 41, and the filtering honeycomb 4 can turn together with the shaft 211 when the second motor is activated.

When the motors and the light tube 311 are activated, air will pass through the filtering honeycomb 4, and ultraviolet light will be emitted from the light tube 311 to shine on the light-catalyst applied over the honeycomb 4, and in turns, air passing through the filtering honeycomb 4 is purified, and germs in the air killed.

From the above description, it can be easily understood that the light-catalyst equipped electric fan of the present invention has advantages as followings:
1. It takes lower cost to manufacture the light tube 311 and the filtering honeycomb 4 than it does to manufacture the above conventional light-catalyst equipped light tube 20.
2. The filtering honeycomb 4 alone can be replaced with a new one when the light-catalyst applied over it loses effect after certain length of time of use because the light tube 311 and the filtering honeycomb 4 are disposed apart, and because the filtering honeycomb 4 can be easily removed and fitted in position. Therefore, there won't be waste of money.
3. Because the light tube 311 and the filtering honeycomb 4 are disposed apart, replacement of the light tube 311 won't cause unwanted position change of the filtering honeycomb 4, and vice versa. Therefore, expected service life of the light tube 311 and the filtering honeycomb 4 won't be reduced, and the present electric fan is economical to use.

1. A light-catalyst equipped electric fan, comprising
a holding part supported on a main body of the fan; the holding part having a locating portion having a plurality of ribs arranged in a radial pattern; the ribs having screw holes formed thereon;
a seat disposed on a front side of the holding part; the seat having through holes thereon;
a light tube for producing light; the light tube being secured to the seat by means of fasteners, which are disposed over the light tube, and which are secured to the seat by screws screwed through the fasteners, the through holes of the seat, and the screw holes of the ribs;
a first motor secured to a rear side of the locating portion;
a fan blade connected to the first motor;
a rear protecting net disposed behind the blade, and secured to the holding part;
a second motor disposed on a front side of the locating portion and having a rotary output shaft connected to an output shaft thereof;
a filtering honeycomb arranged in front of the second motor; the filtering honeycomb being made of paper, and having titanium dioxide applied over it to be used as light catalyst; and
a front protecting net arranged at a front of the electric fan, and connected with the rotary output shaft at a center thereof; the filtering honeycomb being stuck on a rear side of front protecting net.

2. The light-catalyst equipped electric fan as claimed in claim 1, wherein an inner protecting net is disposed behind, and connected with the front protecting net so that the filtering honeycomb is protected between both of the nets; the front protecting net having an annular trench on the edge of a rear side, and screw holes spaced apart along the annular trench; the inner protecting net having a plurality of protrusions on an edge of a front side thereof, and through holes between the protrusions; the protecting nets being joined together with the protrusions being inserted in the annular trench, and with screws being screwed through the holes thereof and screwed into nuts.

3. The light-catalyst equipped electric fan as claimed in claim 1, wherein an ornamental cap is connected to the center of the front protecting net, and connected with the rotary output shaft.
